SEQUENCE LISTING (8 sequences)

SEQ ID NO: 1 - protein, 277 aa, Homo sapiens
MKYLLPTAAAGLLLLAAQPAMAEVQLVESGGGVVRPGGSLRLSCAASGFSFDDYGMSWVRQAPGKGLEWVSGINWNGGSTGYADSVKGRFTISRDNAKNSLYLQMNSLRAEDTAVYYCARMRAPVIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 2 - protein, 278 aa, Homo sapiens
MKYLLPTAAAGLLLLAAQPAMAEVQLVESGGGVVRPGGSLRLSCAASGFTFDLTHPYFWVRQAPGKGLEWVSGINWNGGSTGYADSVKGRFTISRDNAKNSLYLQMNSLRAEDTAVYYCARMRAPVIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 3 - protein, 280 aa, Homo sapiens
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSERAEDTAVYYCARMRAPVIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 4 - protein, 6 aa, Homo sapiens
MRAPVI

SEQ ID NO: 5 - protein, 16 aa, Homo sapiens
GINWNGGSTGYADSVK

SEQ ID NO: 6 - protein, 5 aa, Homo sapiens
DYGMS

SEQ ID NO: 7 - protein, 6 aa, Homo sapiens
LTHPYF

SEQ ID NO: 8 - protein, 8 aa, Homo sapiens
LNPKVKHM

(19) United States
(12) Patent Application Publication
(10) Pub. No.: US 2004/0202665 A1
(43) Pub. Date: Oct. 14, 2004

(54) Compositions and methods for therapeutic treatment

(76) Inventors: Janette Lazarovits, Reut (IL); Abraham Nimrod, Rehovot (IL); Hagit Hoch Mar-Chaim, Aseret (IL); Avigdor Levanon, Rehovot (IL)

Correspondence Address: KENYON & KENYON, ONE BROADWAY, NEW YORK, NY, 10004, US

(21) Appl. No.: 10/610,843

(22) Filed: Jun. 30, 2003

Related U.S. Application Data
(60) Provisional application No. 60/393,453, filed on Jul. 1, 2002.

(51) Int. Cl. A61K 39/395

(52) U.S. Cl. 424/178.1

(57) ABSTRACT

The present invention relates to compositions utilizing an agent and an antibody, or fragment thereof. In these compositions, the agents, including agents such as anti-cancer, anti-metastasis, anti-leukemia, anti-disease, anti-adhesion, anti-thrombosis, anti-restenosis, anti-autoimmune, anti-aggregation, anti-bacterial, anti-viral, and anti-inflammatory agents, can be complexed or combined with or conjugated to the antibodies, or fragments thereof. In addition, the agent and/or the antibody, or fragment thereof, can be present in the composition in a sub-clinical amount, which is an amount that is less than the amount of the agent generally found to be clinically effective when the agent is administered alone. Preferably, in these compositions of the present invention, the agent is an anthracycline or a derivative thereof, e.g., doxorubicin (adriamycin) or a derivative thereof.

### FIELD OF THE INVENTION

The present invention relates to therapeutic and diagnostic compositions and methods, utilizing agents and antibodies, which may have anti-cancer activity, anti-metastatic activity, anti-leukemia activity, anti-viral activity, anti-infection activity, and/or activity against other diseases, such as inflammatory diseases, diseases involving abnormal or pathogenic adhesion, thrombosis and/or restenosis, diseases involving abnormal or pathogenic aggregation, and autoimmune diseases, cardiovascular diseases, such as myocardial infarction, retinopathic diseases, diseases caused by sulfated tyrosine-dependent protein-protein interactions, and diseased cells generally.

### BACKGROUND OF THE INVENTION

Antibodies, Phage Display, and Tissue Targeting

Tissue-selective targeting of therapeutic agents is an emerging discipline in the pharmaceutical industry. New cancer treatments based on targeting have been designed to increase the specificity and potency of the treatment, while reducing toxicity and enhancing overall efficacy. Mouse monoclonal antibodies (MAbs) to tumor-associated antigens have been employed in an attempt to target toxin, radionucleotide, and chemotherapeutic conjugates to tumors. In addition, differentiation antigens, such as CD19, CD20, CD22 and CD25, have been exploited as cancer specific targets in treating hematopoietic malignancies. Although extensively studied, this approach has several limitations. One limitation is the difficulty of isolating appropriate monoclonal antibodies that display selective binding. A second limitation is the need for high antibody immunogenicity as a prerequisite for successful antibody isolation. A third limitation is that the final product comprises non-human sequences, which gives rise to an immune response to the non-human material (e.g., human anti-mouse antibody-HAMA response). The HAMA response often results in a shorter serum half-life and prevents repetitive treatments, thus diminishing the therapeutic value of the antibody. This latter limitation has stimulated interest both in engineering chimeric or humanized monoclonal antibodies of murine origin, and in discovering human antibodies. Another limitation of this approach is that it enables the isolation of only a single antibody species directed against only known and purified antigens. Moreover, this method is not selective insofar as it allows for the isolation of antibodies against cell surface markers that are present on normal, as well as on malignant, cells.

There are many factors that influence the therapeutic efficacy of MAbs for treating cancer. These factors include specificity of antigen expression on tumor cells, level of expression, antigenic heterogeneity and accessibility of the tumor mass. Leukemia and lymphoma have been generally more responsive to treatment with antibodies than solid tumors, such as carcinomas. MAbs rapidly bind to leukemia and lymphoma cells in the bloodstream and easily penetrate to malignant cells in lymphatic tissue, thus making lymphoid tumors excellent candidates for MAb-based therapy. An ideal system entails identifying a MAb that recognizes a marker on the cell surface of stem cells that are producing malignant progeny cells.

Phage libraries are used to select random single chain Fvs (scFvs) that bind to isolated, pre-determined target proteins such as antibodies, hormones and receptors. In addition, the use of antibody display libraries in general, and phage scFv libraries in particular, facilitates an alternative means of discovering unique molecules for targeting specific, yet unrecognized and undetermined, cell surface moieties.

Leukemia, lymphoma, and myeloma are cancers that originate in the bone marrow and lymphatic tissues and are involved in uncontrolled growth of cells. Acute lymphoblastic leukemia (ALL) is a heterogeneous disease that is defined by specific clinical—and immunological characteristics. Like other forms of ALL, the definitive cause of most cases of B-cell ALL (B-ALL) is not known although, in many cases, the disease results from acquired genetic alterations in the DNA of a single cell, causing it to become abnormal and multiply continuously. Prognosis for patients afflicted with B-ALL is significantly worse than for patients with other leukemias, both in children and in adults.

Acute Myelogenous Leukemia (AML) is a heterogeneous group of neoplasms with a progenitor cell that, under normal conditions, gives rise to terminally differentiated cells of the myeloid series (erythrocytes, granulocytes, monocytes, and platelets). As in other forms of neoplasia, AML is associated with acquired genetic alterations that result in replacement of normally differentiated myeloid cells with relatively undifferentiated blasts, exhibiting one or more type of early myeloid differentiation. AML generally evolves in the bone marrow and, to a lesser degree, in the secondary hematopoietic organs. AML primarily affects adults, peaking in incidence between the ages of 15-40, but it is also known to affect both children and older adults. Nearly all patients with AML require treatment immediately after diagnosis to achieve clinical remission, in which there is no evidence of abnormal levels of circulating undifferentiated blast cells.

To date, a variety of monoclonal antibodies has been developed that induce cytolytic activity against tumor cells. A humanized version of the monoclonal antibody MuMAb4D5, directed to the extracellular domain of P185-growth factor receptor (HER2)— was approved by the FDA and is being used to treat human breast cancer (U.S. Pat. Nos. 5,821,337 and 5,720,954). Following binding, the antibody is capable of inhibiting tumor cell growth that is dependent on the HER2 growth factor receptor. In addition, a chimeric antibody against CD20, which causes rapid depletion of peripheral B cells, including those associated with lymphoma, was recently approved by the FDA (U.S. Pat. No. 5,843,439). The binding of this antibody to target cells results in complement-dependent lysis. This product has recently been approved and is currently being used in the clinic to treat low-grade B-cell non-Hodgkin's lymphoma.

Several other humanized and chimeric antibodies are under development or are in clinical trials. In addition, a humanized Ig that specifically reacts with CD33 antigen, expressed both on normal myeloid cells as well as on most types of myeloid leukemic cells, was conjugated to the anti-cancer drug calicheamicin, CMA-676 (Sievers et al., Blood Supplement, 308, 504a (1997)). This conjugate, known as the drug MYLOTARG®, has recently received FDA approval (Caron et al., Cancer Supplement, 73, 1049-1056 (1994)). In light of its cytolytic activity, an additional anti-CD33 antibody (HumM195), currently in clinical trials, was conjugated to several cytotoxic agents, including the gelonin toxin (McGraw et al., Cancer Immunol. Immunother, 39, 367-374 (1994)) and radioisotopes ¹³¹I (Caron et al., Blood 83, 1760-1768 (1994)), ⁹⁰Y (Jurcic et al., Blood Supplement, 92, 613a (1998)) and ²¹³Bi (Humm et al., Blood Supplement, 38:231P (1997)).

A chimeric antibody against the leukocyte antigen CD45 (cHuLym3) is in clinical studies for treatment of human leukemia and lymphoma (Sun et al., Cancer Immunol. Immunother., 48, 595-602 (2000)). In in vitro assays, specific cell lysis was observed in ADCC (antibody dependent cell-mediated cytotoxicity) assays (Henkart, Immunity, 1, 343-346 (1994); Squier and Cohen, Current Opin. Immunol., 6,447-452 (1994)).

In contrast to mouse monoclonal humanization and construction of chimeric antibodies, the use of phage display technology enables the isolation of scFvs comprising fully human sequences. A fully human antibody against the human TGFb2 receptor based on an scFv clone derived from phage display technology was recently developed. This scFv, converted into a fully human IgG4 that is capable of competing with the binding of TGFb2 (Thompson et al., J. Immunol Methods, 227, 17-29 (1999)), has strong anti-proliferative activity. This technology, known to one skilled in the art, is more specifically described in the following publications: Smith, Science, 228, 1315 (1985); Scott et al, Science, 249, 386-390 (1990); Cwirla et al., PNAS, 87, 6378-6382 (1990); Devlin et al., Science, 249, 404-406 (1990); Griffiths et al., EMBO J, 13(14), 3245-3260 (1994); Bass et al., Proteins, 8, 309-314 (1990); McCafferty et al., Nature, 348, 552-554(1990); Nissim et al., EMBO J., 13, 692-698 (1994); U.S. Pat. Nos. 5,427,908, 5,432,018, 5,223,409 and 5,403,484, lib.

Ligand for Isolated scFv Antibody Molecules

Platelets, fibrinogen, GPIb, selecting, and P-Selectin Glycoprotein Ligand-1 (PSGL-1) each play an important role in several pathogenic conditions or disease states, such as abnormal or pathogenic inflammation, abnormal or pathogenic immune reactions, autoimmune reactions, metastasis, abnormal or pathogenic adhesion, thrombosis and/or restenosis, and abnormal or pathogenic aggregation. Thus, antibodies that cross-react with platelets and with these molecules would be useful in the diagnosis and treatment of diseases and disorders involving these and other pathogenic conditions.

Platelets

Platelets are well-characterized components of the blood system and play several important roles in hemostasis, thrombosis and/or restenosis, and restenosis. Damage to blood vessel sets in motion a process known as hemostasis, which is characterized by series of sequential events. The initial reaction to damaged blood vessels is the adhesion of platelets to the affected region on the inner surface of the vessel. The next step is the aggregation of many layers of platelets onto the previously adhered platelets, forming the hemostatic plug. This clump of platelets seals the vessel wall. The hemostatic plug is strengthened by the deposition of fibrin polymers. The clot is degraded only when the damage has been repaired.

Importance of Platelets in Metastasis

Tumor metastasis is perhaps the most important factor limiting the survival of cancer patients. Accumulated data indicate that the ability of tumor cells to interact with host platelets represents one of the indispensable determinants of metastasis. Leslie Oleksowicz, Z. M., “Characterization Of Tumor-Induced Platelet Aggregation: The Role Of Immunorelated GPIb And GPIIb/IIIa Expression By MCF-7 Breast Cancer Cells,” Thrombosis Research 79: 261-274 (1995).

It has been demonstrated that the ability of tumor cells to aggregate platelets correlates with the tumor cells' metastasis potential, and inhibition of tumor-induced platelet aggregation has been shown to correlate with the suppression of metastasis in rodent models. It has been demonstrated that tumor cell interaction with platelets involves membrane adhesion molecules and agonist secretion. Expression of immunorelated platelet glycoproteins has been identified on tumor cell lines. It was demonstrated that platelet immunorelated glycoproteins, GPIb, GPIIb/IIIa. GPIb/IX and the integrin α_{ν} subunit are expressed on the surface of breast tumor cell lines. Oleksowicz, Z. M., “Characterization Of Tumor-Induced Platelet Aggregation: The Role Of Immunorelated GPIb And GPIIb/IIIa Expression By MCF-7 Breast Cancer Cells,” Thrombosis Research 79: 261-274 (1995); Kamiyama, M., et al., “Inhibition of platelet GPIIb/IIIa binding to fibrinogen by serum factors: studies of circulating immune complexes and platelet antibodies in patients with hemophilia, immune thrombocytopenic purpura, human immunodeficiency virus-related immune thrombocytopenic purpura, and systemic lupus erythematosus,” J Lab Clin Med 117(3): 209-17 (1991).

Gasic (J. T. B. Gasic et al., Proc. Natl. Acad. Sci. USA 61:46-52 (1968)) and coworkers showed that antibody-induced thrombocytopenia markedly reduced the number and volume of metastases produced by CT26 colon adenocarcinoma, Lewis lung carcinoma, and B16 melanoma. Karpatkin, S., et al., “Role of adhesive proteins in platelet tumor interaction in vitro and metastasis formation in vivo,” J. Clin. Invest. 81(4): 1012-9 (1988); Clezardin, P., et al., “Role of platelet membrane glycoproteins Ib/IX and IIb/IIIa, and of platelet alpha-granule proteins in platelet aggregation induced by human osteosarcoma cells,” Cancer Res. 53(19): 4695-700 (1993). Furthermore, a single polypeptide chain (60 kd) was found to be expressed on surface membrane of HEL cells which is closely related to GPIb and corresponds to an incompletely or abnormally O-glycosylated GPIbα subunit. Kieffer, N., et al., “Expression of platelet glycoprotein Ib alpha in HEL cells,” J. Biol. Chem. 261(34): 15854-62 (1986).

GPIb Complex

Each step in the process of hemostasis requires the presence of receptors on the platelet surface. One receptor that is important in hemostasis is the glycoprotein Ib-IX complex (also known as CD42). This receptor mediates adhesion (initial attachment) of platelets to the blood vessel wall at sites of injury by binding von Willebrand factor (vWF) in the subendothelium. It also has crucial roles in two other platelet functions important in hemostasis: (a) aggregation of platelets induced by high shear in regions of arterial stenosis and (b) platelet activation induced by low concentrations of thrombin.

The GPIb-IX complex is one of the major components of the outer surface of the platelet plasma membrane. The GPIb-IX complex comprises three membrane-spanning polypeptides- a disulfide-linked 130 kDa α-chain and 25 kDa β-chain of GPIb and noncovalently associated GPIX (22 kDa). All four units are presented in equimolar amounts on the platelet membrane, for efficient cell-surface expression and function of CD42 complex, indicating that proper assembly of the three subunits into a complex is required for full expression on the plasma membrane. The α-chain of GPIb consists of three distinct structural domains: (1) a globular N-terminal peptide domain containing leucine-rich repeat sequences and Cys-bonded flanking sequences; (2) a highly glycosylated mucin-like macroglycopeptide domain; and (3) a membrane-associated C-terminal region that contains the disulfide bridge to GPIbβ and transmembrane and cytoplasmic sequences.

Several lines of evidence indicate that the vWF and thrombin-binding domain of the GPIb-IX complex reside in a globular region that encompasses approximately 300 amino acids at the amino terminus of GPIbα. The human platelets GPIb-IX complex is a key membrane receptor mediating both platelet function and reactivity. Recognition of subendothelial-bound vWF by GPIb allows platelets to adhere to damaged blood vessels. Further, binding of vWF to GPIbα also induces platelet activation, which may involve the interaction of a cytoplasmic domain of the GPIb-IX with cytoskeleton or phospolipase A2. Moreover, GPIbα contains a high-affinity binding site for α-thrombin, which, by an as-yet poorly defined mechanism, facilitates platelet activation.

The N-terminal globular domain of GPIbα contains a cluster of negatively charged amino. Several lines of evidence indicate that, in transfected CHO cells expressing GPIb-IX complex and in platelet GPIbα, the three tyrosine residues contained in this domain (Tyr-276, Tyr-278, and Tyr-279) undergo sulfation.

Protein Sulfation

Protein sulfation is a widespread posttranslational modification that involves enzymatic covalent attachment of sulfate, either to sugar side chains or to the polypeptide backbone. This modification occurs in the trans-Golgi compartment and, therefore affects only protein that traverses this compartment. Such proteins include secretory proteins, proteins targeted for granules, and the extracellular regions of plasma membrane proteins. Tyrosine is an amino acid residue presently known to undergo sulfation. J. W. Kehoe et al., Chemistry and Biol 7: R57-R61 (2000). Other amino acids, for example threonine, may perhaps also undergo sulfation, particularly in diseased cells.

A number of proteins have been found to be tyrosine-sulfated, but the presence of three or more sulfated tyrosines in a single polypeptide, as was found on GPIb, is not common. GPIbα (CD42), which is expressed by platelets and megakaryocytes mediates platelet attachment to and rolling on subendothelium via binding with vWF, also contains numerous negative charges at its N-terminal domain. Such a highly acidic and hydrophilic environment is thought to be a prerequisite for sulfation because tyrosylprotein sulfotransferase specifically recognizes and sulfates tyrosines adjacent to acidic amino residues. J. R. Bundgaard et al., JBC 272:21700-21705 (1997). Full sulfation of the acidic region of GPIbα yields a region with remarkable density of negative charge—13 negative charges within a 19 amino acid stretch, making it a candidate site for electrostatic interaction with other proteins.

Selectins and PSGL-1

The P- , E-, and L-Selectins are a family of adhesion molecules that, among other functions, mediate rolling of leukocytes on vascular endothelium. P-Selectin is stored in granules in platelets and is transported to the surface after activation by thrombin, histamine, phorbol ester, or other stimulatory molecules. P-Selectin is also expressed on activated endothelial cells. E-Selectin is expressed on endothelial cells, and L-Selectin is expressed on neutrophils, monocytes, T cells, and B cells.

PSGL-1 (also called CD162) is a mucin glycoprotein ligand for P-Selectin, E-Selectin, and L-Selectin. PSGL-1 is a disulfide-linked homodimer that has a PACE (Paired Basic Amino Acid Converting Enzymes) cleavage site. PSGL-1 also has three potential tyrosine sulfation sites followed by approximately 15 decamer repeats that are high in proline, serine, and threonine. The extracellular portion of PSGL-1 contains three N-linked glycosylation sites and has numerous sialylated, fucosylated O-linked oligosaccharide branches. K. L. Moore et al., JBC 118:445-456 (1992). Most of the N-glycan sites and many of the O-glycan sites are occupied. The structures of the O-glycans of PSGL-1 from human HL-60 cells have been determined. A subset of these O-glycans are core-2, sialylated and fucosylated structures that are required for binding to selectins. Tyrosine sulfation of an amino-terminal region of PSGL-I is also required for binding to P-Selectin and L-Selectin. Further, there is an N-terminal propeptide that is probably cleaved post-translationally.

PSGL-1 has 361 residues in HL60 cells, with a 267 residue extracellular region, a 25 residue trans-membrane region, and a 69 residue intracellular region. The sequence encoding PSGL-I is in a single exon, so alternative splicing should not be possible. However, PSGL-1 in HL60 cells, and in most cell lines, has 15 consecutive repeats of a 10 residue consensus sequences present in the extracellular region, but there are 14 and 16 repeats of this sequence, as well, in polymorphonuclear leukocytes, monocytes, and several other cell lines, including most native leukocytes. PSGL-1 forms a disulfide-bonded homodimer on the cell surface. V. Afshar-Kharghan et al., Blood 97:3306-3312 (2001).

PSGL-1 is expressed on neutrophils as a dimer, with apparent molecular weight of both 250 kDa and 160 kDa, whereas on HL60 the dimeric form is ˜220 kDa. When analyzed under reducing conditions, each subunit is reduced by half Differences in molecular mass may be due to polymorphisms in the molecule caused by the presence of different numbers of decamer repeats. Leukocyte Typing VI. Edited by T. Kishimoto et al. (1997).

PSGL-1 is expressed on most blood leukocytes, such as neutrophils, monocytes, leukocytes, subset of B cells, and all T cells and mediates rolling of neutrophils on P-selectin. Leukocyte Typing VI. Edited by T. Kishimoto et al. (1997). PSGL-1 may also mediate neutrophil-neutrophil interaction via binding with L-Selectin, thereby mediating inflammation. Snapp, et al., Blood 91(1): 154-64 (1998).

PSGL-1 mediates rolling of leukocytes on activated endothelium, on activated platelets, and on other leukocytes and inflammatory sites.

A commercially available monoclonal antibody to human PGSL-1, KPL1, was generated and shown to inhibit the interactions between PGSL-I and P-selectin and between PGSL-1 and L-selectin. The KPL1 epitope was mapped to the tyrosine sulfation consensus motif of PGSL-1 (YEYLDYD). KPL1 recognizes only this particular epitope and does not cross-react with sulfated epitopes present on other cells, such as B-CLL cells, AML cells, metastatic cells, multiple myeloma cells, and the like.

Leukocyte rolling is important in inflammation, and interaction between P-Selectin (expressed by activated endothelium and on platelets, which may be immobilized at sites of injury) and PSGL-1 is instrumental for tethering and rolling of leukocytes on vessel walls. Ramachandran et al., PNAS 98(18): 10166-71 (2001); Afshar-Kharghan, et al., Blood 97(10): 3306-7 (2001).

Cell rolling is also important in metastasis, and P- and E-Selectin on endothelial cells is believed to bind metastatic cells, thereby facilitating extravasation from the blood stream into the surrounding tissues.

Platelets are also involved in the process of metastasis; when metastatic cancer cells enter the blood stream, multicellular complexes composed of platelets and leukocytes coating the tumor cells are formed. These complexes, which may be referred to as microemboli, aid the tumor cells in evading the immune system. The coating of tumor cells by platelets requires expression of P-selectin by the platelets.

Treatment with heparin, an inhibitor of P- and L-Selectin inhibits tumor cell-platelet interaction. Pretreatment of tumor cells with O-sialoglycoprotease, which removes sialylated, fucosylated mucin ligands, also inhibited tumor cell-platelet complex formation. In vivo experiments indicate that either of these treatments results in greater monocyte association with circulating tumor cells, suggesting that reducing platelet binding increases access by immune cells to circulating tumor cells. Varki and Varki, Braz. J. Biol. Res. 34(6): 711-7 (2001).

PSGL-1 and GPIb share structural similarity, having mucin-like, highly glycosylated ligand binding regions. Afshar-Kharghan, et al., Blood 97(10): 3306-7 (2001).

PSGL-1 has been found on all leukocytes: neutrophils, monocytes, lymphocytes, activated peripheral T-cells, granulocytes, eosinophils, platelets and on some CD34 positive stem cells and certain subsets of B-cells. P-Selectin is selectively expressed on activated platelets and endothelial cells. Interaction between P-Selectin and PSGL-1 promotes rolling of leukocytes on vessel walls, and abnormal accumulation of leukocytes at vascular sites results in various pathological inflammations. Stereo-specific contributions of individual tyrosine sulfates on PSGL-1 are important for the binding of P-Selectin to PSGL-1. Charge is also important for binding: reducing NaCl (from 150 to 50 mM) enhanced binding (Kd˜75 nM). Tyrosine-sulfation on PSGL-1 enhances, but is not ultimately required for PSGL-1 adhesion on P-Selectin. PSGL-1 tyrosine sulfation supports slower rolling adhesion at all shear rates and supports rolling adhesion at much higher shear rates. (Rodgers S D, et al., Biophys J 81: 2001-9 (2001)).

Fibrinogen

There are two forms of normal human fibrinogen: fibrinogen γ major and fibrinogen γ prime minor variant, each of which is found in normal individuals. Normal fibrinogen, which is the more abundant form (comprising ˜90% of the fibrinogen found in the body), is composed of two identical 55 kDa alpha (α) chains, two identical 95 kDa beta (β) chains, and two identical 49.5 kDa gamma (γ) chains. Normal variant fibrinogen, which is the less abundant form (comprising ˜10% of the fibrinogen found in the body), is composed of two identical 55 kDa alpha (α) chains, two identical 95 kDa beta (β) chains, one 49.5 kDa gamma (γ) chain, and one 50.5 kDa gamma prime (γ′) chain. The gamma and gamma prime chains are both coded for by the same gene, with alternative splicing occurring at the 3′ end. Normal gamma chain is composed of amino acids 1-411. Normal variant gamma prime chain is composed of 427 amino acids: amino acids 1-407 are the same as those in the normal gamma chain, and amino acids 408-427 are VRPEHPAETEYDSLYPEDDL. This region is normally occupied with thrombin molecules.

Fibrinogen is converted into fibrin by the action of thrombin in the presence of ionized calcium to produce coagulation of the blood. Fibrin is also a component of thrombi, and acute inflammatory exudates.

Platelets, and molecules (such as fibrinogen, GPIb, selecting, and PSGL-1) that play important roles in cell-cell interactions, cell-matrix interactions, platelet-platelet interactions, platelet-cell interactions, platelet-matrix interactions, cell rolling and adhesion, and hemostasis also play important roles in pathogenic conditions or disease states, such as abnormal or pathogenic inflammation, abnormal or pathogenic immune reactions, autoimmune reactions, metastasis, abnormal or pathogenic adhesion, thrombosis and/or restenosis, and abnormal or pathogenic aggregation. Thus, antibodies that cross-with react with platelets and with these molecules are useful in the diagnosis and treatment of diseases and disorders involving these and other pathogenic conditions.

It is an object of the present invention to provide compositions of an antibody, or fragment thereof, and an agent, including an agent such as an anti-cancer, anti-metastasis, anti-leukemia, anti-disease, anti-adhesion, anti-thrombosis, anti-restenosis, anti-autoimmune, anti-aggregation, anti-bacterial, anti-viral, or anti-inflammatory agent.

It is another object of the present invention to provide methods utilizing agents and antibodies, or fragments thereof, to treat various conditions, including those related to cell rolling; inflammation; auto-immune disease; metastasis; growth and/or replication of tumor cells; mortality of tumor cells; growth and/or replication of leukemia cells; mortality rate of leukemia cells; susceptibility of diseased cells to damage by anti-disease agents; susceptibility of tumor cells to damage by anti-cancer agents; susceptibility of leukemia cells to damage by anti-leukemia agents; the number of tumor cells in a patient having a tumor or cancer; and the number of leukemia cells in a patient having leukemia.

It is a further object of the present invention to provide methods of therapeutic treatment using an agent and an antibody, or fragment thereof, wherein the agent and/or the antibody, or fragment thereof, is present in the composition in an amount that is less than the amount of the agent generally found to be clinically effective when the agent is administered alone.

These and other objectives of the invention are provided herein.

### SUMMARY OF THE INVENTION

The present invention provides a composition having an agent and an antibody, or fragment thereof. The present inventive compositions are such that the agent can be complexed with the antibody, or fragment thereof; the agent can be combined with the antibody, or fragment thereof; or the agent can be conjugated to an antibody, or fragment thereof. The antibodies, or fragments thereof, of the present inventive compositions can be present as complexes or aggregates of one or more antibodies, or fragments.

In the present inventive compositions, the agent and/or the antibody, or fragment thereof, can be present in a sub-clinical amount. This sub-clinical amount can be insufficient to alter effectively susceptibility to agents, especially susceptibility of diseased cells. The agent can be an anti-cancer, anti-metastasis, anti-leukemia, anti-disease, anti-adhesion, anti-thrombosis, anti-restenosis, anti-autoimmune, anti-aggregation, anti-bacterial, anti-viral, or anti-inflammatory agent. Preferably, the agent is an anthracycline or a derivative thereof, which can be doxorubicin, daunorubicin, idarubicin, morpholinodoxorubicin, morpholinodaunorubicin, methoxymorpholinyldoxorubicin, or derivatives or combinations thereof. Most preferably, the agent is doxorubicin or a derivative thereof.

Moreover, the sub-clinical amount of the agent can be insufficient to inhibit effectively cell rolling, inflammation, auto-immune disease, thrombosis, restenosis, metastasis, or survival, growth, and/or replication of tumor cells or leukemia cells. In addition, the sub-clinical amount of the agent can be insufficient to inhibit effectively an increase in the number of tumor cells in a patient having a tumor or inhibit an increase in the number of leukemia cells in a patient having leukemia. Also, the sub-clinical amount of the agent can be insufficient to decrease effectively the number of tumor cells in a patient having a tumor or decrease effectively the number of leukemia cells in a patient having leukemia. Additionally, the sub-clinical amount of the agent can be insufficient to increase effectively mortality of tumor cells or leukemia cells, increase effectively susceptibility of tumor cells to damage by anti-cancer agents, or increase effectively susceptibility of leukemia cells to damage by anti-leukemia agents. Finally, the sub-clinical amount of the agent can be insufficient to inhibit effectively cell-cell, cell-matrix, platelet-matrix, platelet-platelet, and/or cell-platelet complex formation, aggregation, or adhesion.

The antibody, or fragment thereof, of the present inventive compositions can have the binding capabilities of an scFv antibody fragment of SEQ ID NO:1, SEQ ID NO:2, or SEQ ID NO:3. In addition, the antibody, or fragment thereof, can have the binding capabilities of a peptide or polypeptide, wherein the peptide or polypeptide has a first hypervariable region having SEQ ID NO:4. Such a peptide or polypeptide also can further have a second hypervariable region having SEQ ID NO:5 and/or a third hypervariable region having SEQ ID NO:6, SEQ ID NO:7, or SEQ ID NO:8. The antibody, or fragment thereof, can be an scFv or an Fab fragment.

The composition of the present invention can also have an agent and an antibody, or fragment thereof, wherein the antibody, or fragment thereof, binds to a peptide or polypeptide epitope of about 3 to about 126 amino acid residues in length, which peptide or polypeptide epitope has at least 2 acidic amino acids and at least one sulfated tyrosine residue. Additionally, the compositions of the present invention can have an agent and an antibody, or fragment thereof, wherein the antibody, or fragment thereof, binds to at least two different molecules selected from the group consisting of PSGL-1, fibrinogen gamma prime (y′), GPIbα, heparin, lumican, complement compound 4 (CC4), interalpha inhibitor, and prothrombin. Also additionally, the compositions of the present invention can have an agent and an antibody, or fragment thereof, wherein the antibody, or fragment thereof, binds to at least two different molecules selected from the group consisting of PSGL-1, fibrinogen gamma prime (y′), GPIbα, heparin, lumican, complement compound 4 (CC4), interalpha inhibitor, and prothrombin and that binds to at least one cell type selected from the group consisting of B cell leukemia cells, B-CLL cells, AML cells, multiple myeloma cells, and metastatic cells. Moreover, the compositions of the present invention can have an agent and an antibody, or fragment thereof, wherein the antibody, or fragment thereof, cross-reacts with two or more epitopes, each epitope comprising one or more sulfated tyrosine residues and at least one cluster of two or more acidic amino acids.

In the compositions of the present invention, the antibody, or fragment thereof, can be coupled to or complexed or combined with a vehicle or carrier that is coupled to or complexed or combined with more than one agent. Such a vehicle or carrier can be selected from the group consisting of dextran, lipophilic polymers, hydrophilic polymers, HPMA, and liposomes. Preferably, the vehicle or carrier is a doxorubicin-decorated liposome. Alternatively, preferably, the vehicle or carrier is polyethylene glycol (PEG) or dextran.

The present invention also provides various methods of administering a composition of the present invention to a patient in need thereof. For example, the present invention also provides methods of ameliorating the effects of a disease, preventing a disease, treating a disease, or inhibiting the progress of a disease by administering to a patient in need thereof any of the present inventive compositions. The present invention provides a method of inhibiting cell rolling, inflammation, auto-immune disease, thrombosis, restenosis, metastasis, survival, growth and/or replication of tumor cells, growth and/or replication of leukemia cells, increase in the number of tumor cells in a patient having a tumor, or increase in the number of leukemia cells in a patient having leukemia. Also, the present invention provides methods of increasing the mortality rate of tumor cells, the mortality rate of leukemia cells, the susceptibility of diseased cells to damage by anti-disease agents, the susceptibility of tumor cells to damage by anti-cancer agents, or the susceptibility of leukemia cells to damage by anti-cancer agents. Further, the present invention provides methods of decreasing the number of tumor cells in a patient having a tumor, the number of leukemia cells in a patient having leukemia. Finally, the present invention provides methods of inhibiting cell-cell, cell-matrix, platelet-matrix, platelet-platelet, and/or cell-platelet complex formation, aggregation, or adhesion.

The present invention also provides a method of therapeutic treatment by administering to a patient in need thereof (i) an antibody, or fragment thereof, and (ii) an agent, wherein one or both of the antibody, or fragment thereof, and/or the agent is administered in a sub-clinical amount. In this method, the agent and antibody, or fragment thereof, can be conjugated, complexed, or administered in combination. Further, the agent can be administered separately, prior or subsequent to the antibody, or fragment thereof, or in any other sequence or arrangement and vice versa.

### DEFINITIONS

Antibodies (Abs), or immunoglobulins (IgGs), are protein molecules that bind to antigen. They are composed of units of four polypeptide chains (2 heavy and 2 light) linked together by disulfide bonds. Each of the chains has a constant and variable region. They can be divided into five classes, IgG, IgM. IgA, IgD, and IgE, based on their heavy chain component. The IgG class encompasses several sub-classes including, but not restricted to, IgG₁, IgG₂, IgG₃, and IgG₄. Immunoglobulins are produced in vivo by B lymphocytes and recognize a particular foreign antigenic determinant and facilitate clearing of that antigen.

Antibodies may be produced and used in many forms, including antibody complexes. As used herein, the term “antibody complex” or “antibody complexes” is used to mean a complex of one or more antibodies with another antibody or with an antibody fragment or fragments, or a complex of two or more antibody fragments. Examples of antibody fragments include Fv, Fab, F(ab′)₂, F(ab′), Fc, and Fd fragments.

As used herein in the specification and in the claims, an Fv is defined as a molecule that is made up of a variable region of a heavy chain of a human antibody and a variable region of a light chain of a human antibody, which may be the same or different, and in which the variable region of the heavy chain is connected, linked, fused or covalently attached to, or associated with, the variable region of the light chain. The Fv can be a single chain Fv (scFv) or a disulfide stabilized Fv (dsFv). An scFv is comprised of the variable domains of each of the heavy and light chains of an antibody, linked by a flexible amino-acid polypeptide spacer, or linker. The linker may be branched or unbranched. Preferably, the linker is 0-15 amino acid residues, and most preferably the linker is (Gly₄Ser)₃.

The Fv molecule itself is comprised of a first chain and a second chain, each chain comprising a first, second and third hypervariable region. The hypervariable loops within the variable domains of the light and heavy chains are termed Complementary Determining Regions (CDR). There are CDR1, CDR2 and CDR3 regions in each of the heavy and light chains. These regions are believed to form the antigen binding site and can be specifically modified to yield enhanced binding activity. The most variable of these regions in nature being the CDR3 region of the heavy chain. The CDR3 region is understood to be the most exposed region of the Ig molecule and as shown and provided herein is the site primarily responsible for the selective and/or specific binding characteristics observed.

A fragment of an Fv molecule is defined as any molecule smaller than the original Fv that still retains the selective and/or specific binding characteristics of the original Fv. Examples of such fragments include but are limited to (I) a minibody, which comprises a fragment of the heavy chain only of the Fv, (2) a microbody, which comprises a small fractional unit of antibody heavy chain variable region (PCT Application No. PCT/IL99/00581), (3) similar bodies comprising a fragment of the light chain, and (4) similar bodies comprising a functional unit of a light chain variable region.

As used herein the term “Fab fragment” is a monovalent antigen-binding fragment of an immunoglobulin. A Fab fragment is composed of the light chain and part of the chain.

A F(ab′)₂ fragment is a bivalent antigen binding fragment of an immunoglobulin obtained by pepsin digestion. It contains both light chains and part of both heavy chains.

A Fc fragment is a non-antigen-binding portion of an immunoglobulin. It contains the carboxy-terminal portion of heavy chains and the binding sites for the Fc receptor.

A Fd fragment is the variable region and first constant region of the heavy chain of an immunoglobulin.

Polyclonal antibodies are the product of an immune response and are formed by a number of different B-lymphocytes. Monoclonal antibodies are derived from a single cell.

A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed or attached at one or both ends.

The term “epitope” is used herein to mean the antigenic determinant or antigen site that interacts with an antibody, antibody fragment, antibody complex or a complex comprising a binding fragment thereof or T-cell receptor. The term epitope is used interchangeably herein with the terms ligand, domain, and binding region.

Selectivity is herein defined as the ability of a targeting molecule to choose and bind one cell type or cell state from a mixture of cell types or cell states, all cell types or cell states of which may be specific for the targeting molecule.

The term “affinity” as used herein is a measure of the binding strength (association constant) between a receptor (e.g., one binding site on an antibody) and a ligand (e.g., antigenic determinant). The strength of the sum total of noncovalent interactions between a single antigen-binding site on an antibody and a single epitope is the affinity of the antibody for that epitope. Low affinity antibodies bind antigen weakly and tend to dissociate readily, whereas high-affinity antibodies bind antigen more tightly and remain bound longer. The term “avidity” differs from affinity because the former reflects the valence of the antigen-antibody interaction.

Specificity of antibody-antigen interaction: Although the antigen-antibody reaction is specific, in some cases antibodies elicited by one antigen can cross-react with another unrelated antigen. Such cross-reactions occur if two different antigens share a homologous or similar structure, epitope, or an anchor region thereof, or if antibodies specific for one epitope bind to an unrelated epitope possessing similar structure conformation or chemical properties.

A platelet is a disc-like cytoplasmic fragment of a megakaryocyte that is shed in the marrow sinus and subsequently are circulating in the peripheral blood stream. Platelets have several physiological functions including a major role in clotting. A platelet contains granules in the central part and peripherally, clear protoplasm, but no definite nucleus.

Agglutination as used herein means the process by which suspended bacteria, cells, discs, or other particles of similar size are caused to adhere and form into clumps. The process is similar to precipitation but the particles are larger and are in suspension rather than being in solution.

The term aggregation means a clumping of platelets induced in vitro, and thrombin and collagen, as part of a sequential mechanism leading to the formation of a thrombus or hemostatic plug.

Conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polar, non-polar) such that the substitutions do not substantially in a major way alter peptide, polypeptide or protein characteristics (e.g., charge, IEF, affinity, avidity, conformation, solubility) or activity. Typical substitutions that may be performed for such conservative amino acid substitution may be among the groups of amino acids as follows:

glycine (G), alanine (A), valine (V), leucine (L) and isoleucine (I)

aspartic acid (D) and glutamic acid (E)

alanine (A), serine (S) and threonine (T)

histidine (H), lysine (K) and arginine (R)

asparagine (N) and glutamine (Q)

phenylalanine (F), tyrosine (Y) and tryptophan (W)

Conservative amino acid substitutions can be made in, as well as, flanking the hypervariable regions primarily responsible for the selective and/or specific binding characteristics of the molecule, as well as other parts of the molecule, e.g., variable heavy chain cassette. Additionally or alternatively, modification can be accomplished by reconstructing the molecules to form full-size antibodies, diabodies (dimers), triabodies (timers) and/or tetrabodies (tetramers) or to form minibodies or microbodies.

A phagemid is defined as a phage particle that carries plasmid DNA. Phagemids are plasmid vectors designed to contain an origin of replication from a filamentous phage, such as m113 of fd. Because it carries plasmid DNA, the phagemid particle does not have sufficient space to contain the full complement of the phage genome. The component that is missing from the phage genome is information essential for packaging the phage particle. In order to propagate the phage, therefore, it is necessary to culture the desired phage particles together with a helper phage strain that complements the missing packaging information.

A promoter is a region on DNA at which RNA polymerase binds and initiates transcription.

A phage display library (also termed phage peptide/antibody library, phage library, or peptide/antibody library) comprises a large population of phage (generally 10⁸-10⁹), each phage particle displaying a different peptide or polypeptide sequence. These peptide or polypeptide fragments may constructed to be of variable length. The displayed peptide or polypeptide can be derived from, but need not be limited to, human antibody heavy or light chains.

A pharmaceutical composition refers to a formulation which comprises a peptide or polypeptide of the invention and a pharmaceutically acceptable carrier, excipient or diluent thereof.

A pharmaceutical agent refers to an agent that is useful in the prophylactic treatment or diagnosis of a mammal including, but not restricted to, a human, bovine, equine, porcine, murine, canine, feline, or any other warm-blooded animal. The pharmaceutical agent is selected from the group comprising radioisotope, toxin, oligonucleotide, recombinant protein, antibody fragment, and anti-cancer agent. Examples of such pharmaceutical agents include, but are not limited to anti-viral agents including acyclovir, ganciclovir and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diflitox, subreum, WinRho SDF, defibrotide, and cyclophosphamide; and anti-adhesion/anti-aggregation agents including limaprost, clorcromene, and hyaluronic acid.

An anti-leukemia agent is an agent with anti-leukemia activity. For example, anti-leukemia agents include agents that inhibit or halt the growth of leukemic or immature pre-leukemic cells, agents that kill leukemic or pre-leukemic, agents that increase the susceptibility of leukemic or pre-leukemic cells to other anti-leukemia agents, and agents that inhibit metastasis of leukemic cells. In the present invention, an anti-leukemia agent may also be agent with anti-angiogenic activity that prevents, inhibits, retards or halts vascularization of tumors.

The expression pattern of a gene can be studied by analyzing the amount of gene product produced under various conditions, at specific times, in various tissues, etc. A gene is considered to be “over expressed” when the amount of gene product is higher than that found in a normal control, e.g., non-diseased control.

A given cell may express on its surface a protein having a binding site (or epitope) for a given antibody, but that binding site may be exist in a cryptic form (e.g., be sterically hindered or be blocked, or lack features needed for binding by the antibody) in the cell in a state, which may be called a first stage (stage I). Stage I may be, for example, a normal, healthy, non-diseased status. When the epitope exists in cryptic form, it is not recognized by the given antibody, i.e., there is no binding of the antibody to this epitope or to the given cell at stage 1. However, the epitope may be exposed by, e.g., undergoing modifications itself, or being unblocked because nearby or associated molecules are modified or because a region undergoes a conformational change. Examples of modifications include changes in folding, changes in post-translational modifications, changes in phospholipidation, changes in sulfation, changes in glycosylation, and the like. Such modifications may occur when the cell enters a different state, which may be called a second stage (stage II). Examples of second states, or stages, include activation, proliferation, transformation, or in a malignant status. Upon being modified, the epitope may then be exposed, and the antibody may bind.

Peptido-mimetics are small molecules, peptides, polypeptides, lipids, polysaccharides or conjugates thereof that have the same functional effect or activity of another entity such as an antibody.

### BRIEF DESCRIPTION OF THE DRAWING

FIG. 1 graphically represents percent survival of MOLT-4 tumor-bearing mice as a function of time (days) following administration of doxorubicin and Y1 alone, sequentially, or in combination.

### DETAILED DESCRIPTION OF THE INVENTION

The present invention relates to compositions and methods involving an agent and an antibody, or fragment thereof. The compositions of the present invention can be such that one or more antibodies, or fragments thereof, are aggregated, associated, complexed, or combined with or conjugated, fused, or linked to one or more of various agents, such as drugs, toxins, and radioactive isotopes with, optionally, a pharmaceutically effective carrier, to form drug-peptide complexes, compositions, or conjugates having anti-disease and/or anti-cancer activity. Such complexes, combinations, conjugates, may also be used for diagnostic purposes. Moreover, the agent and/or the antibody can be present in the composition in a sub-clinical amount. By sub-clinical amount is meant an amount that is less than the amount of the agent and/or antibody generally found to be clinically optimally effective when the agent and/or antibody is administered alone. A sub-clinical amount can also mean an amount that is less than the amount required of the agent and/or antibody generally found to elicit a defined clinical response. It should be appreciated that sub-clinical is not intended to mean that the agent and/or antibody is clinically ineffective when administered according to the present inventive compositions and methods.

In a preferred embodiment, the sub-clinical amount of the agent can be insufficient to effectively alter susceptibility to agents, particularly susceptibility of diseased cells. For example, the sub-clinical amount of the agent can be insufficient to inhibit effectively cell rolling, inflammation, auto-immune disease, thrombosis, restenosis, metastasis, or growth and/or replication of tumor cells or leukemia cells. Also, the sub-clinical amount of the agent can be insufficient to inhibit effectively an increase in the number of tumor cells in a patient having a tumor or inhibit an increase in the number of leukemia cells in a patient having leukemia. The sub-clinical amount of the agent also can be insufficient to decrease effectively the number of tumor cells in a patient having a tumor or decrease effectively the number of leukemia cells in a patient having leukemia. In addition, the sub-clinical amount of the agent can be insufficient to increase effectively mortality of tumor cells or leukemia cells, increase effectively susceptibility of tumor cells to damage by anti-cancer agents, or increase effectively susceptibility of leukemia cells to damage by anti-leukemia agents. Finally, the sub-clinical amount of the agent can be insufficient to inhibit effectively cell-cell, cell-matrix, platelet-matrix, platelet-platelet, and/or cell-platelet complex formation, aggregation, or adhesion.

Preferably, the present inventive compositions have an antibody, or fragment thereof, having the binding capabilities of an scFv antibody fragment of SEQ ID NO: 1, SEQ ID NO:2, or SEQ ID NO:3. The scFv fragment of SEQ ID NO: 1 has been designated Y1, the scFv fragment of SEQ ID NO:2 has been designated Y17, and the scFv fragment of SEQ ID NO:3 has been designated L32. These antibodies were identified by screening a human antibody phage library, which has diversity only in the heavy chain CDR3 regions. In the case of the human scFv Y1 and Y17 antibodies, fixed human platelets were screened in order to identify antibodies that bind platelets. L32 was screened against a leukemia cell to select specific antibodies that recognize leukemia cell surface determinants, wherein the specific receptor was not previously known or characterized. Using this same method, another antibody, L31, was identified.

Previously, antibodies useful in the present inventive composition were identified in U.S. application Ser. Nos. 10/032,423; 10/032,037; 10/029,988; 10/029,926; 09/751,181; and 60/258,948 and International Application Nos. PCT/US01/49442 and PCT/US01/49440 using the same phage library. Specific examples of antibodies disclosed in these applications include the Y1 and Y17 antibodies. The antibodies disclosed in these applications were discovered to specifically bind to an epitope found on proteins of the hematopoetic cells, which is sulfated at an N-terminal tyrosine and thought to be involved in cell migration, e.g., tumor metastasis.

The epitope for Y1 antibody is located between amino acids 272 and 285 on glycocalicin, one of the subunits of the CD42 complex in which there is cluster of negatively charged amino acids, resulting from the sulfated groups, which are essential for the binding of Y1 to glycocalicin. In addition, Y1 binds the N-terminal of PSGL-1, which is a receptor for E, L- and P-selectins, containing sulfated tyrosine residues accompanied by a cluster of negatively charged amino acids. Although the Y1 antibody binds to several molecules, such as the glycocalicin molecule on platelets, fibrinogen-gamma prime, the complement compound 4 of human plasma, and the PSGL-1 molecule on KG-1 cells, its affinity to primary leukemia cells derived from either AML or multiple myeloma (MM) patients is several magnitudes higher relative to the previously mentioned epitopes.

In addition, the L32 and L31 antibodies were disclosed in U.S. Application No. 60/______, entitled “L32 Antibodies and Uses Thereof,” which was filed Jul. 1, 2002. Both the L32 antibody and the antibodies disclosed in the Y1/Y17 applications bind leukemic cells, although L32 binds to leukemic cells with approximately five times greater affinity than Y1. While the L32 and Y1/Y17 antibodies were all isolated from a common germ line (DP32) and L32 appears to bind the same sulfated epitope as Y1/Y17, L32 does not bind platelets and, moreover, does not affect platelet aggregation.

The sulfated epitopes previously identified as binding to the preferred antibodies of the present invention are characterized by the presence of sulfated moieties, such as sulfated tyrosine residues or sulfated carbohydrate or lipid moieties, preferably within a cluster of two or more acidic amino acids, which are found on ligands and receptors that play important roles in such diverse processes as inflammation, immune reactions, infection, autoimmune reactions, metastasis, adhesion, thrombosis and/or restenosis, cell rolling, and aggregation. Such epitopes are also found on diseased cells, such as B cell leukemia cells, B-CLL cells, AML cells, multiple myeloma cells, and metastatic cells. These epitopes are useful targets for the therapeutic mediation of these processes and for diagnostic procedures.

Preferably, the antibodies of the present inventive compositions binds different molecules or epitopes involved in inflammation, such as PSGL-1, fibrinogen gamma prime (y′), GPIb, heparin, lumican, complement compound 4 (CC4), interalpha inhibitor, and prothrombin. Also preferably, the antibodies bind to an epitope present on at least one cell type involved in inflammation or tumorigenesis, including B-CLL cells, T-ALL cells, AML cells, B-leukemia cells, multiple myeloma cells, and metastatic cells. Further preferably, the antibodies the present inventive compositions bind to epitopes on a lipid, carbohydrate, peptide, glycolipid, glycoprotein, lipoprotein, and/or lipopolysaccharide molecule. Such epitopes preferably have at least one sulfated moiety. Alternatively, but also preferably, the antibodies cross-react with two or more epitopes, each epitope having one or more sulfated tyrosine residues, and at least one cluster of two or more acidic amino acids, an example of which is PSGL-1.

It is the hypervariable regions of the antibodies of the present invention that participate in forming the antigen binding sites. The antigen-binding site is complementary to the structure of the epitopes to which the antibodies bind; therefore these binding sites are referred to as complementarity-determining regions (CDRs). There are three CDRs on each light and heavy chain of an antibody (CDR1, CDR2, and CDR3), each located on the loops that connect the β strands of the V_{H} and V_{L} domains. The most variable of these regions is the CDR3 region of the heavy chain. The CDR3 region is understood to be the most exposed region of the Ig molecule and, as provided herein, has a central role in determining the selective and/or specific binding characteristics observed.

In one preferred embodiment of the present inventive compositions, the antibody, or fragment thereof, has a first hypervariable region (CDR3) of SEQ ID NO:4. In addition, or alternatively, the antibody, or fragment thereof, has a second hypervariable region (CDR2) of SEQ ID NO:5. Also in addition, or alternatively, the antibody, fragment thereof, has a third hypervariable region (CDR1) of SEQ ID NO:6, SEQ ID NO:7, or SEQ ID NO:8.

According to the present invention, CDRs may also be inserted into cassettes to produce antibodies. A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed, or attached at one or both ends. The amino acid sequence of the cassette may ostensibly be fixed, whereas the replaced, inserted, or attached sequence can be highly variable. The cassette can be comprised of several domains, each of which encompasses a function crucial to the final construct. The cassette of a particular embodiment of the present invention comprises, from the N-terminus, framework region 1 (FR1), CDR1, framework region 2 (FR2), CDR2, framework region 3 (FR3), and framework region 4 (FR4). In an embodiment of the invention, it is possible to replace distinct regions within the cassette. For example, the CDR2 and CDR1 hypervariable regions of the cassette may be replaced or modified by non-conservative or, preferably, conservative amino acid substitutions.

For all of the amino acid sequences of ≦25 amino acid residues described and detailed herein (e.g., CDR regions, CDR flanking regions), it is to be understood and considered as a further embodiment of the invention that these amino acid sequences include within their scope one or two amino acid substitution(s) and that preferably the substitutions are conservative amino acid substitutions. For all of the amino acid sequences of >25 amino acid residues described and detailed herein, it is to be understood and considered as an embodiment of the invention that these amino acid sequences include within their scope an amino acid sequence with ≧90% sequence similarity to the original sequence (Altschul et al., Nucleic Acids Res. 25: 3389-402 (1997)). Similar or homologous amino acids are defined as non-identical amino acids which display similar properties, e.g., acidic, basic, aromatic, size, positively or negatively charged, polarity, non-polarity.

Percent amino acid similarity or homology or sequence similarity is determined by comparing the amino acid sequences of two different peptides or polypeptides. Antibody sequences were determined by DNA sequencing. The two sequences are aligned, usually by use of one of a variety of computer programs designed for the purpose, and amino acid residues at each position are compared. Amino acid identity or homology is then determined. An algorithm is then applied to determine the percentage amino acid similarity. It is generally preferable to compare amino acid sequences, due to the greatly increased sensitivity to detection of subtle relationships between the peptide, polypeptide or protein molecules. Protein comparison can take into account the presence of conservative amino acid substitutions, whereby a mismatch may yet yield a positive score if the non-identical amino acid has similar physical and/or chemical properties (Altschul et al. (1997), supra).

In an embodiment of the invention, the three hypervariable regions of each of the light and heavy chains can be interchanged between the two chains and among the three-hypervariable sites within and/or between chains.

The present invention provides for a peptide or polypeptide having an antibody, or fragment thereof, a construct thereof, or a construct of a fragment. According to the present invention, antibodies include IgG, IgA, IgD, IgE, or IgM antibodies. The IgG class encompasses several sub-classes including IgG₁, IgG₂, IgG₃, and IgG₄.

Antibodies may be provided in many forms, such as fragments, complexes, and multimers. According to the present invention, antibody fragments include Fv, scFv, dsFv, Fab, Fab₂, and Fd molecules. Smaller antibody fragments, such as fragments of Fvs and fragments of Fabs, are also included in the term “fragments”, as long as they retain the binding characteristics of the original antibody or larger fragment. Examples of such fragments would be (1) a minibody, which comprises a fragment of the heavy chain only of the Fv, (2) a microbody, which comprises a small fractional unit of antibody heavy chain variable region (International Application No. PCT/IL99/00581), (3) similar bodies having a fragment of the light chain, and (4) similar bodies having a functional unit of a light chain variable region. Constructs include, for example, multimers such as diabodies, triabodies, and tetrabodies. The phrases “antibody, or fragment thereof, or complex having an antibody, or fragment thereof” and “antibody or fragment” are intended to encompass all of these molecules, as well as derivatives and homologs, mimetics, and variants thereof, unless it is specified otherwise or indicated otherwise based on context and/or knowledge in the art.

It has been established that scFv penetrate tissues and are cleared from the blood more rapidly than a full size antibody because they are smaller in size (Adams et al., Br. J. Cancer 77: 1405-12 (1988); Hudson, Curr. Opin. Immunol. 11(5): 548-557 (1999); Wu et al., Tumor Targeting 4: 47 (1999)). Thus, scFv are often employed in diagnostics involving radioactive labels such as tumor imaging to allow for a more rapid clearance of the radioactive label from the body. A number of cancer-targeting scFv multimers have recently undergone pre-clinical evaluation for in vivo stability and efficacy (Adams et al. (1988), supra; Wu (1999), supra).

Typically, scFv monomers are designed with the C-terminal end of the V_{H} domain tethered by a polypeptide linker to the N-terminal residue of the V_{L}. Optionally an inverse orientation is employed: the C-terminal end of the V_{L} domain is tethered to the N-terminal residue of V_{H} through a polypeptide linker (Power et al., J Immun. Meth. 242: 193-204 (2000)). The polypeptide linker is typically around fifteen amino acids in length. When the linker is reduced to about three to seven amino acids, the scFvs can not fold into a functional Fv domain and instead associate with a second scFv to form a diabody. Further reducing the length of the linker to less than three amino acids forces the scFv association into trimers or tetramers, depending on the linker length, composition and Fv domain orientations. (Powers (2000), supra).

Recently, it has been discovered that multivalent antibody fragments such as scFv dimers, trimers, and tetramers often provide higher affinity over the binding of the parent antibody to the target. This higher affinity offers potential advantages including improved pharmaco-kinetics for tumor targeting applications. Additionally, in studying P-Selectin and its ligand PSGL-1, which are involved in tethering and rolling of leukocytes, scientists have concluded that cells expressing dimeric forms of PSGL-1 established more stable rolling adhesions because of this higher binding affinity. These adhesions are more sheer resistant and exhibited less fluctuation in rolling velocities (Ramachandran et al., PNAS, 98(18): 10166-71 (2001)).

The greater binding affinity of these multivalent forms may be beneficial in diagnostics and therapeutic regimens. For example, an scFv may be employed as a blocking agent to bind a target receptor and thus block the binding of the “natural” ligand. In such instances, it is desirable to have a higher affinity association between the scFv and the receptor to decrease chances for disassociation, which may allow an undesirable binding of the natural ligand to the target. In addition, this higher affinity may be useful when the target receptors are involved in adhesion and rolling or when the target receptors are on cells present in areas of high sheer flow, such as platelets.

Once an antibody, fragment, or construct having desired binding capabilities has been selected and/or developed, it is well within the ability of one skilled in the art using the guidance provided herein to produce constructs and fragments which retain the characteristics of the original antibody. For example, full antibody molecules, Fv fragments, Fab fragments, Fab₂ fragments, dimers, trimers, and other constructs can be made which retain the desired characteristics of the originally selected or developed antibody, fragment, or construct.

If it is desired to substitute amino acids, but still retain the characteristics of an antibody or fragment, it is well within the skill in the art to make conservative amino acid substitutions. Modifications such as complexing or combining with or conjugating to pharmaceutical or diagnostic agents may also be made to antibodies or fragments without altering their binding characteristics. Other modifications, such as those made to produce more stable antibodies or fragments may also be made to antibodies or fragments without altering their specificity. For example, peptoid modification, semipeptoid modification, cyclic peptide modification, N terminus modification, C terminus modification, peptide bond modification, backbone modification, and residue modification may be performed. It is also within the ability of the skilled worker following the guidance of the present specification to test the modified antibodies or fragments to assess whether their binding characteristics have been changed.

Likewise, it is within the ability of the skilled worker using the guidance provided herein to alter the binding characteristics of an antibody, fragment, or construct to obtain a molecule with more desirable characteristics. For example, once an antibody having desirable properties is identified, random or directed mutagenesis may be used to generate variants of the antibody, and those variants may be screened for desirable characteristics.

Using conventional methods known in the art, one of skill would also be able to determine addition antibodies, or fragments thereof, that have the binding capabilities useful in the present inventive compositions. For example, additional antibodies can be isolated using the biopanning methods described herein, wherein a molecule or cell that binds to fixed human platelets or leukemic cells is used to screen a particular phage display library, particularly a library prepared from a leukemia, lymphoma, or myeloma patient.

Using conventional methods known in the art, one of skill also would be able to determine antibodies, or fragments thereof, that have the binding capabilities of an scFv antibody fragment of SEQ ID NO: 1, SEQ ID NO:2, or SEQ ID NO:3. Additional antibodies that bind different molecules or epitopes involved in inflammation, such as PSGL-1, fibrinogen gamma prime (γ′), GP1b, heparin, lumican, complement compound 4 (CC4), interalpha inhibitor, and prothrombin, can also be determined using convention methods, which are known in the art. Using conventional methods, one of skill in the art also would be able to determine additional antibodies that bind to an epitope present on at least one cell type involved in inflammation or tumorigenesis, including B-CLL cells, T-ALL cells, AML cells, B-leukemia cells, multiple myeloma cells, and metastatic cells. Moreover, antibodies that bind to epitopes on a lipid, carbohydrate, peptide, glycolipid, glycoprotein, lipoprotein, and/or lipopolysaccharide molecule, wherein the epitopes preferably have at least one sulfated moiety can be determined using conventional methods. Alternatively, conventional methods can also be used to determine antibodies that cross-react with two or more epitopes, each epitope having one or more sulfated tyrosine residues, and at least one cluster of two or more acidic amino acids, an example of which is PSGL-1. For example, binding data can be determined using biosensor analysis, e.g., using a commercial biosensor, BIACORE (Piscataway, N.J.) (Myszka, J. Mol. Recognition, 12: 279-84 (1999); Malmborg & Borrebaeck, J. Immunol. Meth., 183: 7-13 (1999)).

The present invention provides for scFv antibodies. As used herein, an scFv is defined as a molecule which is made up of a variable region of a heavy chain of a human antibody and a variable region of a light chain of a human antibody, which may be the same or different, and in which the variable region of the heavy chain is connected, linked, fused, or covalently attached to, or associated with, the variable region of the light chain.

An scFv construct may be a multimer (e.g., dimer, trimer, tetramer, and the like) of scFv molecules that incorporate one or more of the hypervariable domains of the antibody. All scFv derived constructs and fragments retain enhanced binding characteristics so as to bind selectively and/or specifically to a target cell in favor of other cells. The binding selectivity and/or specificity is primarily determined by hypervariable regions. The antibodies of the subject invention can be constructed to fold into multivalent Fv forms, which may improve binding affinity and specificity and increased half-life in blood.

Mulitvalent forms of scFv have been designed and produced by others. One approach has been to link two scFvs with linkers. Another approach involves using disulfide bonds between two scFvs for the linkage. The simplest approach to production of dimeric or trimeric Fv was reported by Holliger et al., PNAS 90: 6444-48 (1993) and Kortt et al., Protein Eng. 10: 423-33 (1997). One such method was designed to make dimers of scFvs by adding a sequence of the FOS and JUN protein region to form a leucine zipper between them at the c-terminus of the scFv (Kostelny et al., J mmunol. 148(5): 1547-53 (1992); De Kruif et al., J Biol. Chem. 271(13): 7630-34 (1996)). Another method was designed to make tetramers by adding a streptavidin coding sequence at the c-terminus of the scFv. Streptavidin is composed of 4 subunits, so when the scFv-streptavidin is folded, 4 subunits accommodate themselves to form a tetramer (Kipriyanov et al., Hum Antibodies Hybridomas 6(3): 93-101 (1995)). In yet another method, to make dimers, trimers, and tetramers, a free cysteine is introduced in the protein of interest. A peptide-based cross linker with variable numbers (2 to 4) of maleimide groups was used to cross link the protein of interest to the free cysteines (Cochran et al., Immunity 12(3): 241-50 (2000)).

In this system, the phage library (as described herein above) can be designed to display scFvs, which can fold into the monovalent form of the Fv region of an antibody. Further, and also discussed herein above, the construct is suitable for bacterial expression. The genetically engineered scFvs comprise heavy chain and light chain variable regions joined by a contiguously encoded 15 amino acid flexible peptide spacer. The preferred spacer is (Gly₄Ser)₃. The length of this spacer, along with its amino acid, constituents provides for a nonbulky spacer, which allows the V_{H} and the V_{L} regions to fold into a functional Fv domain that provides effective binding to its target.

Varying the length of the spacers is yet another preferred method of forming dimers, trimers, and triamers (often referred to in the art as diabodies, triabodies, and tetrabodies, respectively). Dimers are formed under conditions where the spacer joining the two variable chains of an scFv is shortened to generally 5-12 amino acid residues. This shortened spacer prevents the two variable chains from the same molecule from folding into a functional Fv domain. Instead, the domains are forced to pair with complimentary domains of another molecule to create two binding domains. In a preferred method, a spacer of only 5 amino acids (Gly₄Ser) was used for diabody construction. This dimer can be formed from two identical scFvs, or from two different populations of scFvs and retain the selective and/or specific enhanced binding activity of the parent scFv(s), and/or show increased binding strength or affinity.

In a similar fashion, triabodies are formed under conditions where the spacer joining the two variable chains of an scFv is shortened to generally less than 5 amino acid residues, preventing the two variable chains from the same molecule from folding into a functional Fv domain. Instead, three separate scFv molecules associate to form a trimer. In a preferred method, triabodies were obtained by completely removing this flexible spacer. The triabody can be formed from three identical scFvs, or from two or three different populations of scFvs, and retain the selective and/or specific enhanced binding activity of the parent scFv(s), and/or show increased binding strength or affinity.

Tetrabodies are similarly formed under conditions where the spacer joining the two variable chains of an scFv is shortened to generally less than 5 amino acid residues, preventing the two variable chains from the same molecule from folding into a functional Fv domain. Instead, four separate scFv molecules associate to form a tetramer. The tetrabody can be formed from four identical scFvs, or from 1-4 individual units from different populations of scFvs and should retain the selective and/or specific enhanced binding activity of the parent scFv(s), and/or show increased binding strength or affinity. Whether triabodies or tetrabodies form, under conditions where the spacer is generally less than 5 amino acid residues long, depends on the amino acid sequence of the particular scFv(s) in the mixture and the reaction conditions.

Antibodies, fragments thereof or constructs thereof peptides, polypeptides, proteins, and fragments and constructs thereof can be produced in either prokaryotic or eukaryotic expression systems. Methods for producing antibodies and fragments in prokaryotic and eukaryotic systems are well-known in the art.

A eukaryotic cell system, as defined in the present invention and as discussed, refers to an expression system for producing peptides or polypeptides by genetic engineering methods, wherein the host cell is a eukaryote. A eukaryotic expression system may be a mammalian system, and the peptide or polypeptide produced in the mammalian expression system, after purification, is preferably substantially free of mammalian contaminants. Other examples of a useful eukaryotic expression system include yeast expression systems.

A preferred prokaryotic system for production of the peptide or polypeptide of the invention uses E. coli as the host for the expression vector. The peptide or polypeptide produced in the E. coli system, after purification, is substantially free of E. coli contaminating proteins. Use of a prokaryotic expression system may result in the addition of a methionine residue to the N-terminus of some or all of the sequences provided for in the present invention. Removal of the N-terminal methionine residue, after peptide or polypeptide production to allow for full expression of the peptide or polypeptide, can be performed as is known in the art, one example being with the use of Aeromonas aminopeptidase under suitable conditions (U.S. Pat. No. 5,763,215).

Antibodies and fragments, according to the present invention, may also have a tag that may be inserted or attached thereto to aid in the preparation and identification thereof, and in diagnostics. The tag can later be removed from the molecule. Examples of useful tags include: AU1, AU5, BTag, c-myc, FLAG, Glu-Glu, HA, His6, HSV, HTTPHH, IRS, KT3, Protein C, S-TAG®, T7, V5, and VSV-G (Jarvik and Telmer, Ann. Rev. Gen., 32, 601-18 (1998)). The tag is preferably c-myc or KAK.

Any suitable agent can be used in the compositions of the present invention. Such agents generally have anti-cancer, anti-metastasis, anti-leukemia, anti-disease, anti-adhesion, anti-thrombosis, anti-restenosis, anti-autoimmune, anti-aggregation, anti-bacterial, anti-viral, or anti-inflammatory activity. Accordingly, the agents of the present inventive compositions can be anti-cancer agents, anti-neoplastic agents, anti-viral agents, anti-metastatic agents, anti-inflammatory agents, anti-thrombosis agents, anti-restenosis agents, anti-aggregation agents, anti-autoimmune agents, anti-adhesion agents, anti-cardiovascular disease agents, or other anti-disease agents or pharmaceutical agent. A pharmaceutical agent refers to an agent that is useful in the prophylactic treatment or diagnosis of a mammal including, but not restricted to, a human, bovine, equine, porcine, murine, canine, feline, or any other warm-blooded animal.

Examples of such pharmaceutical agents include, but are not limited to, anti-viral agents including acyclovir, ganciclovir and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid.

Other exemplary pharmaceutical agents include cis-platinum, taxol, calicheamicin, vincristine, cytarabine (Ara-C), cyclophosphamide, prednisone, fludarabine, chlorambucil, interferon alpha, hydroxyurea, temozolomide, thalidomide and bleomycin, and derivatives and combinations thereof. Preferably, the pharmaceutical agent is an anthracycline or a derivative thereof; more preferably, the pharmaceutical agent is doxorubicin (adriamycin), daunorubicin, idarubicin, morpholinodoxorubicin, morpholinodaunorubicin, or methoxymorpholinyldoxorubicin, or derivatives and combinations thereof; and most preferably, the pharmaceutical agent is doxorubicin (adriamycin).

An anti-cancer agent is an agent with anti-cancer activity. For example, anti-cancer agents include agents that inhibit or halt the growth of cancerous or immature pre-cancerous cells, agents that kill cancerous or pre-cancerous, agents that increase the susceptibility of cancerous or pre-cancerous cells to other anti-cancer agents, and agents that inhibit metastasis of cancerous cells. In the present invention, an anti-cancer agent may also be agent with anti-angiogenic activity that prevents, inhibits, retards, or halts vascularization of tumors. Inhibition of growth of a cancer cell includes, for example, the (i) prevention of cancerous or metastatic growth, (ii) slowing down of the cancerous or metastatic growth, (iii) the total prevention of the growth process of the cancer cell or the metastatic process, while leaving the cell intact and alive, (iv) interfering contact of cancer cells with the microenvironment, or (v) killing the cancer cell.

An anti-leukemia agent is an agent with anti-leukemia activity. For example, anti-leukemia agents include agents that inhibit or halt the growth of leukemic or immature pre-leukemic cells, agents that kill leukemic or pre-leukemic, agents that increase the susceptibility of leukemic or pre-leukemic cells to other anti-leukemia agents, and agents that inhibit metastasis of leukemic cells. In the present invention, an anti-leukemia agent may also be agent with anti-angiogenic activity that prevents, inhibits, retards or halts vascularization of tumors. Inhibition of growth of a leukemia cell includes, for example, the (i) prevention of leukemic or metastatic growth, (ii) slowing down of the leukemic or metastatic growth, (iii) the total prevention of the growth process of the leukemia cell or the metastatic process, while leaving the cell intact and alive, (iv) interfering contact of cancer cells with the microenvironment, or (v) killing the leukemia cell.

Examples of anti-disease, anti-cancer, and anti-leukemic agents to which antibodies and fragments of the present invention may usefully be linked include toxins, radioisotopes, and pharmaceuticals. Examples of toxins include gelonin, Pseudomonas exotoxin (PE), PE40, PE38, diphtheria toxin, ricin, or modifications or derivatives thereof. Examples of radioisotopes include gamma-emitters, positron-emitters, and x-ray emitters that may be used for localization and/or therapy, and beta-emitters and alpha-emitters that may be used for therapy. The radioisotopes described previously as useful for diagnostics are also useful for therapeutics. Non-limiting examples of anti-cancer or anti-leukemia pharmaceutical agents include doxorubicin (adriamycin), cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives thereof, and combinations or modifications thereof.

In addition, anti-disease, anti-cancer or anti-leukemia agents can also be growth factor receptor antagonists, which inhibit stimulation of a growth factor receptor by a growth factor receptor ligand, thereby inhibiting growth of cells that express the growth factor receptor. Some examples of growth factor receptors are the receptors for epidermal growth factor (EGFR), vascular endothelial growth factor (VEGFR), platelet-derived growth factor (PDGFR), insulin-like growth factor (IGFR), nerve growth factor (NGFR), and fibroblast growth factor (FGF).

The antibodies and fragments thereof of the subject invention can also be optionally associated, complexed, or combined with or conjugated, fused, or linked to a pharmaceutically effective carrier. Examples of carriers useful in the invention include dextran, lipophilic polymers, such as HPMA, and hydrophilic polymers. Alternatively, decorated liposomes can be used, such as liposomes decorated with scFv Y1 molecules, e.g., Doxil, a commercially available liposome containing large amounts of doxorubicin. Such liposomes can be prepared to contain one or more desired pharmaceutical agents and be admixed with the antibodies of the present invention to provide a high drug to antibody ratio. Preferably, the vehicle or carrier is a doxorubicin-decorated liposome. Alternatively, but also preferably, the vehicle or carrier is the hydrophilic polymer polyethylene glycol (PEG) or dextran.

Alternatively, the link between the antibody or fragment thereof and the pharmaceutical agent may be a direct link. A direct link between two or more neighboring molecules may be produced via a chemical bond between elements or groups of elements in the molecules. The chemical bond can be, for example, an ionic bond, a covalent bond, a hydrophobic bond, a hydrophilic bond, an electrostatic bond, or a hydrogen bond. The bonds can be, for example, amine, carboxy, amide, hydroxyl, peptide, and/or disulfide bonds. The direct link may preferably be a protease resistant bond.

The link between the peptide and the pharmaceutical agent or between the peptide and carrier, or between the carrier and pharmaceutical agent may be via a linker compound. As used herein, in the specification and in the claims, a linker compound is defined as a compound that joins two or more moieties. The linker can be straight-chained or branched. A branched linker compound may be composed of a double-branch, triple branch, or quadruple or more branched compound. Linker compounds useful in the present invention include those selected from the group having dicarboxylic acids, malemido hydrazides, PDPH, carboxylic acid hydrazides, and small peptides.

More specific examples of linker compounds useful, according to the present invention, include: (a) dicarboxylic acids such as succinic acid, glutaric acid, and adipic acid; (b) maleimido hydrazides such as N-[maleimidocaproic acid]hydrazide, 4-[N-maleimidomethyl]cyclohexan-1-carboxylhydrazide, and N-[maleimidoundcanoic acid]hydrazide; (c) PDPH linkers such as (3-[2-pyridyldithio]propionyl hydrazide) conjugated to sulfurhydryl reactive protein; and (d) carboxylic acid hydrazides selected from 2-5 carbon atoms.

Linking via direct coupling using small peptide linkers is also useful. For example, direct coupling between the free sugar of, for example, the anti-cancer drug doxorubicin and an scFv may be accomplished using small peptides. Examples of small peptides include AU1, AU5, BTag, c-myc, FLAG, Glu-Glu, HA, His6, HSV, HTTPHH, IRS, KT3, Protein C, S-TAG®, T7, V5, VSV-G, and KAK.

Antibodies, and fragments thereof, of the present invention may be bound to, conjugated to, complexed with, or otherwise associated with imaging agents (also called indicative markers), such as radioisotopes, and these conjugates can be used for diagnostic and imaging purposes. Kits having such radioisotope-antibody (or fragment) conjugates are provided.

Examples of radioisotopes useful for diagnostics include ¹¹¹indium, ¹¹³indium, ^{99m}rhenium, ¹⁰⁵rhenium, ¹⁰¹rhenium, ^{99m}technetium, ^{121m}tellurium, ^{122m}tellurium, ^{125m}tellurium ¹⁶⁵thulium, ¹⁶⁷thulium ¹⁶⁸thulium ¹²³iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵ruthenium, ¹⁰⁷mercury, ²⁰³mercury, ⁶⁷gallium, and ⁶⁸gallium. Preferred radioactive isotopes, are opaque to X-rays or any suitable paramagnetic ions.

The indicative marker molecule may also be a fluorescent marker molecule. Examples of fluorescent marker molecules include fluorescein, phycoerythrin, or rhodamine, or modifications or conjugates thereof.

Antibodies or fragments conjugated to indicative markers may be used to diagnose or monitor disease states. Such monitoring may be carried out in vivo, in vitro, or ex vivo. Where the monitoring or diagnosis is carried out in vivo or ex vivo, the imaging agent is preferably physiologically acceptable in that it does not harm the patient to an unacceptable level. Acceptable levels of harm may be determined by clinicians using such criteria as the severity of the disease and the availability of other options.

The present invention provides for a diagnostic kit for in vitro analysis of treatment efficacy before, during, or after treatment, having an imaging agent having a peptide of the invention linked to an indicative marker molecule, or imaging agent. The invention further provides for a method of using the imaging agent for diagnostic localization and imaging of a cancer, more specifically a tumor, having the following steps: (a) contacting the cells with the composition; (b) measuring the radioactivity bound to the cells; and hence (c) visualizing the tumor.

Examples of suitable imaging agents include fluorescent dyes, such as FITC, PE, and the like, and fluorescent proteins, such as green fluorescent proteins. Other examples include radioactive molecules and enzymes that react with a substrate to produce a recognizable change, such as a color change.

In one example, the imaging agent of the kit is a fluorescent dye, such as FITC, and the kit provides for analysis of treatment efficacy of cancers, more specifically blood-related cancers, e.g., leukemia, lymphoma, or myeloma. FACS analysis is used to determine the percentage of cells stained by the imaging agent and the intensity of staining at each stage of the disease, e.g., upon diagnosis, during treatment, during remission and during relapse.

The present invention also provides methods of ameliorating the effects of a disease, preventing a disease, treating a disease, or inhibiting the progress of a disease by administering to a patient in need thereof any of the present inventive compositions. For example, the present invention provides a method of inhibiting cell rolling, inflammation, auto-immune disease, thrombosis, restenosis, metastasis, growth and/or replication of tumor cells, growth and/or replication of leukemia cells, increase in the number of tumor cells in a patient having a tumor, or increase in the number of leukemia cells in a patient having leukemia. Also, the present invention provides methods of increasing the mortality rate of tumor cells, the mortality rate of leukemia cells, the susceptibility of diseased cells to damage by anti-disease agents, the susceptibility of tumor cells to damage by anti-cancer agents, or the susceptibility of leukemia cells to damage by anti-cancer agents. Further, the present invention provides methods of decreasing the number of tumor cells in a patient having a tumor, the number of leukemia cells in a patient having leukemia. Finally, the present invention provides methods of inhibiting cell-cell, cell-matrix, platelet-matrix, platelet-platelet, and/or cell-platelet complex formation, aggregation, or adhesion.

The present inventive methods are preferably carried out with one or both of the agent and antibody administered at an amount that is sub-clinical. Accordingly, the present invention provides a method of therapeutic treatment that involves administering to a patient in need thereof (i) an antibody, or fragment thereof, and (ii) an agent, wherein one or both of the antibody, or fragment thereof, and/or the agent is administered in a sub-clinically effective amount. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition. It should be noted, however, that the present invention is not limited to any particular dose.

Moreover, one of skill in the art will appreciate that the methods of the present invention include administration of the agent and antibody in a single administration or multiple administrations, which can be such that the agent is administrated prior to, concurrently with, or subsequent to administration of the antibody, or fragment thereof. The agent can be administered prior to, concurrently with, or subsequent to the antibody, or fragment thereof, or in any other sequence or arrangement and vice versa. As such, the agent and antibody can be present in separate compositions.

The antibodies, constructs, conjugates, combinations, and fragments of the subject invention may be administered to patients in need thereof via any suitable method. Exemplary methods include intravenous, intramuscular, subcutaneous, topical, intratracheal, intrathecal, intraperitoneal, intralymphatic, nasal, sublingual, oral, rectal, vaginal, respiratory, buccal, intradermal, transdermal or intrapleural administration.

For intravenous administration, the formulation preferably will be prepared so that the amount administered to the patient will be an effective amount from about 0.1 mg to about 1000 mg of the desired composition. More preferably, the amount administered will be in the range of about 1 mg to about 500 mg of the desired composition. The compositions of the invention are effective over a wide dosage range, and depend on factors such as the particulars of the disease to be treated, the half-life of the peptide or polypeptide-based pharmaceutical composition in the body of the patient, physical and chemical characteristics of the pharmaceutical agent and of the pharmaceutical composition, mode of administration of the pharmaceutical composition, particulars of the patient to be treated or diagnosed, as well as other parameters deemed important by the treating physician.

Pharmaceutical composition for oral administration may be in any suitable form. Examples include tablets, liquids, emulsions, suspensions, syrups, pills, caplets, and capsules. Methods of making pharmaceutical compositions are well known in the art. See, e.g., Remington, The Science and Practice of Pharmacy, Alfonso R. Gennaro (Ed.) Lippincott, Williams & Wilkins (pub).

The pharmaceutical composition may also be formulated so as to facilitate timed, sustained, pulsed, or continuous release. The pharmaceutical composition may also be administered in a device, such as a timed, sustained, pulsed, or continuous release device.

The pharmaceutical composition for topical administration can be in any suitable form, such as creams, ointments, lotions, patches, solutions, suspensions, lyophylizates, and gels.

Compositions comprising antibodies, constructs, conjugates, combination, and fragments of the subject invention may comprise conventional pharmaceutically acceptable diluents, excipients, carriers, and the like. Tablets, pills, caplets and capsules may include conventional excipients such as lactose, starch and magnesium stearate. Suppositories may include excipients such as waxes and glycerol. Injectable solutions comprise sterile pyrogen-free media such as saline, and may include buffering agents, stabilizing agents or preservatives. Conventional enteric coatings may also be used.

The following examples are set forth to aid in understanding the invention but are not intended and should not be construed, to limit its scope in any way. Although specific reagents and reaction conditions are described, modifications can be made that are encompassed by the scope of the invention. The following examples, therefore, are provided to further illustrate the invention. All references mentioned or described herein are incorporated in their entirety.

### EXAMPLES

### Example 1

The present example examines the interaction between chemotherapy treatment and Y1 in the MOLT-4 tumor-bearing mice.

Initially, SCID mice (Jackson) were pretreated with 100 mg/kg CTX (Cytoxan-cyclophosphamid for injection, Mead Johnson). Five days after CTX injection, MOLT-4 (T leukemia) cells were inoculated intravenously (i.v.) through the tail vein with 2×10⁷ cells. Mice were randomly divided into 5 treatment groups (13 per group), and they were treated, beginning 5 days after cell inoculation, as indicated in the table below. Tumor-bearing mice were treated for two weeks with sub-optimal dose of doxorubicin (Dox), in combination with Y1 given either concomitantly or after the Dox course of treatment. The response to the therapies was monitored as survival. As described above, the experimental groups were as follows:

TABLE 1

| Group | Administered materials (i.v.) | Frequency of treatment |
|---|---|---|
| Control | No further treatment | — |
| Dox | 2 mg Dox/kg mouse | 2 doses, 1x/wk, beginning day |
| | | 5 post inoculation |
| YI | 0.1 mg Y1 scFv/mouse | 6 doses, 3x/wk, beginning day |
| | | 5 post inoculation |
| Sequential | 2 mg Dox/kg mouse and then | 2 doses, 1x/wk, beginning day |
| therapy | 0.1 mg Y1 scFv/mouse | 5 post inoculation followed by |
| | | 6 doses, 3x/wk, |
| Combined | 2 mg Dox/kg mouse + 0.1 | 2 doses, 1x/wk, beginning day |
| therapy | mg Y1 scFv/mouse | 5 post inoculation concomi- |
| | | tantly with 6 doses, 3x/wk, |

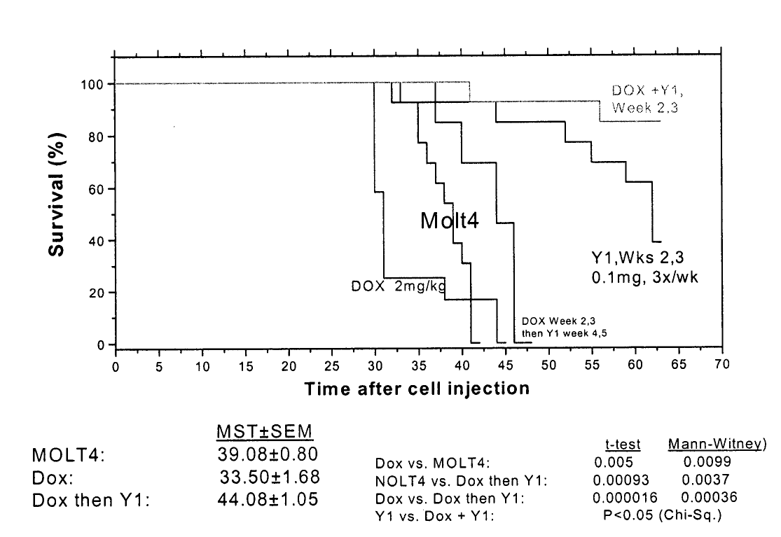

The results in FIG. 1 indicate that treatment with a sub-optimal dose of Dox, alone, had a negative effect on the survival of tumor-bearing mice (the mean survival time—MST is 33.5±1.68 days), relative to the control group (MTS 39.08±0.8 days). However, the survival of mice sequentially treated with Dox, followed by Y1, was highly significantly prolonged. Although Y1 alone had a dramatic effect on the survival rate of tumor-bearing mice, combination of sub-clinically optimal Dox+Y1 had the best effect. The significantly increased survival rate appears to be the result of a synergistic effect between chemotherapy and the Y1 antibody.

We claim:

1. A composition comprising an agent and an antibody, or fragment thereof.

2. The composition of claim 1, wherein the agent is complexed with the antibody, or fragment thereof.

3. The composition of claim 1, wherein the agent is combined with the antibody, or fragment thereof.

4. The composition of claim 1, wherein the agent is conjugated to the antibody, or fragment thereof.

5. The composition of claim 1, wherein the agent is present in a sub-clinical amount.

6. The composition of claim 5, wherein the sub-clinical amount of the agent is insufficient to alter effectively the susceptibility of diseased cells by anti-disease agents.

7. The composition of claim 5, wherein the sub-clinical amount of the agent is insufficient to inhibit effectively cell rolling, inflammation, auto-immune disease, thrombosis, restenosis, metastasis, or growth and/or replication of tumor cells or leukemia cells.

8. The composition of claim 5, wherein the sub-clinical amount of the agent is insufficient to inhibit effectively an increase in the number of tumor cells in a patient having a tumor or inhibit an increase in the number of leukemia cells in a patient having leukemia.

9. The composition of claim 5, wherein the sub-clinical amount of the agent is insufficient to decrease the number of tumor cells in a patient having a tumor or decrease the number of leukemia cells in a patient having leukemia.

10. The composition of claim 5, wherein the sub-clinical amount of the agent is insufficient to increase mortality of tumor cells or leukemia cells, increase susceptibility of tumor cells to damage by anti-cancer agents, or increase susceptibility of leukemia cells to damage by anti-leukemia agents.

11. The composition of claim 5, wherein the sub-clinical amount of the agent is insufficient to inhibit effectively cell-cell, cell-matrix, platelet-matrix, platelet-platelet, and/or cell-platelet complex formation, aggregation, or adhesion.

12. The composition of claim 1, wherein the antibody, or fragment thereof, is present in a sub-clinical amount.

13. The composition of claim 1, wherein the antibody, or fragment thereof, has the binding capabilities of an scFv antibody fragment of SEQ ID NO: 1, SEQ ID NO:2, or SEQ ID NO:3.

14. The composition of claim 1, wherein the antibody, or fragment thereof, has the binding capabilities of a peptide or polypeptide, wherein the peptide or polypeptide comprises a first hypervariable region having SEQ ID NO:4.

15. The composition of claim 14, wherein the peptide or polypeptide further comprises a second hypervariable region having SEQ ID NO:5 and/or a third hypervariable region having SEQ ID NO:6, SEQ ID NO:7, or SEQ ID NO:8.

16. The composition of claim 1, wherein the antibody, or fragment thereof, is an scFv or an Fab fragment.

17. A composition comprising an agent and an antibody, or fragment thereof, wherein the antibody, or fragment thereof, binds to a peptide or polypeptide epitope of about 3 to about 126 amino acid residues in length, wherein the peptide or polypeptide epitope has at least 2 acidic amino acids and at least one sulfated tyrosine residue.

18. A composition comprising an agent and an antibody, or fragment thereof, wherein the antibody, or fragment thereof, binds to at least two different molecules selected from the group consisting of PSGL-1, fibrinogen gamma prime (y′), GPIbα, heparin, lumican, complement compound 4 (CC4), interalpha inhibitor, and prothrombin.

19. A composition comprising an agent and an antibody, or fragment thereof, wherein the antibody, or fragment thereof, binds to at least two different molecules selected from the group consisting of PSGL-1, fibrinogen gamma prime (y′), GPIbα, heparin, lumican, complement compound 4 (CC4), interalpha inhibitor, and prothrombin and that binds to at least one cell type selected from the group consisting of B cell leukemia cells, B-CLL cells, AML cells, multiple myeloma cells, and metastatic cells.

20. A composition comprising an agent and an antibody, or fragment thereof, wherein the antibody, or fragment thereof, cross-reacts with two or more epitopes, each epitope comprising one or more sulfated tyrosine residues and at least one cluster of two or more acidic amino acids.

21. The composition of claim 1, wherein the agent is selected from the group consisting of anti-cancer, anti-metastasis, anti-leukemia, anti-disease, anti-adhesion, anti-thrombosis, anti-restenosis, anti-autoimmune, anti-aggregation, anti-bacterial, anti-viral, and anti-inflammatory agents.

22. The composition of claim 21, wherein the agent is an anti-viral agent selected from the group consisting of acyclovir, ganciclovir and zidovudine.

23. The composition of claim 21, wherein the agent is an anti-thrombosis/anti-restenosis agent selected from the group consisting of cilostazol, dalteparin sodium, reviparin sodium, and aspirin.

24. The composition of claim 21, wherein the agent is an anti-inflammatory agent selected from the group consisting of zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid.

25. The composition of claim 21, wherein the agent is an anti-autoimmune agent selected from the group consisting of leflunomide, denileukin diflitox, subreum, WinRho SDF, defibrotide, and cyclophosphamide.

26. The composition of claim 21, wherein the agent is an anti-adhesion/anti-aggregation agent selected from the group consisiting of limaprost, clorcromene, and hyaluronic acid.

27. The composition of claim 21, wherein the agent is selected from the group consisting of toxins, radioisotopes, and pharmaceutical agents.

28. The composition of claim 27, wherein the toxin is selected from the group consisting of gelonin, Pseudomonas exotoxin (PE), PE40, PE38, ricin, and modifications and derivatives thereof.

29. The composition of claim 27, wherein the radioisotope is selected from the group consisting of gamma-emitters, positron-emitters, x-ray emitters, beta-emitters, and alpha-emitters.

30. The composition of claim 27, wherein the radioisotope is selected from the group consisting of ¹¹¹indium, ¹¹³indium, ^{99m}rhenium, ¹⁰⁵rhenium, ¹⁰¹rhenium, ^{99m}technetium, ^{121m}tellurium, ^{122m}tellurium, ^{125m}tellurium ¹⁶⁵thulium, ¹⁶⁷thulium ¹⁶⁸thulium ¹²³iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵ruthenium, ¹⁰⁷mercury, ²⁰³mercury, ⁶⁷gallium and ⁶⁸gallium.

31. The composition of claim 27, wherein the pharmaceutical agent is selected from the group consisting of cis-platinum, taxol, calicheamicin, vincristine, cytarabine (Ara-C), cyclophosphamide, prednisone, fludarabine, chlorambucil, interferon alpha, hydroxyurea, temozolomide, thalidomide and bleomycin, and derivatives and combinations thereof.

32. The composition of claim 27, wherein the pharmaceutical agent is an anthracycline or a derivative thereof.

33. The composition of claim 32, wherein the pharmaceutical agent is selected from the group consisting of doxorubicin, daunorubicin, idarubicin, morpholinodoxorubicin, morpholinodaunorubicin, methoxymorpholinyldoxorubicin, and derivatives and combinations thereof.

34. The composition of claim 32, wherein the pharmaceutical agent is doxorubicin or a derivative thereof.

35. The composition of claim 1, wherein an antibody, or fragment thereof, is coupled to or complexed or combined with a vehicle or carrier that is coupled to or complexed or combined with more than one agent.

36. The composition of claim 35, wherein the vehicle or carrier is selected from the group consisting of dextran, lipophilic polymers, hydrophilic polymers, HPMA, and liposomes.

37. The composition of claim 36, wherein the vehicle or carrier is a doxorubicin-decorated liposome.

38. The composition of claim 36, wherein the vehicle or carrier is polyethylene glycol (PEG) or dextran.

39. A method of inhibiting cell rolling comprising administering to a patient in need thereof a composition of claim 1.

40. A method of inhibiting inflammation comprising administering to a patient in need thereof a composition of claim 1.

41. A method of inhibiting auto-immune disease comprising administering to a patient in need thereof a composition of claim 1.

42. A method of inhibiting thrombosis comprising administering to a patient in need thereof a composition of claim 1.

43. A method of inhibiting restenosis comprising administering to a patient in need thereof a composition of claim 1.

44. A method of inhibiting metastasis comprising administering to a patient in need thereof a composition of claim 1.

45. A method of inhibiting growth and/or replication of tumor cells comprising administering to a patient in need thereof, a composition of claim 1.

46. A method of increasing the mortality rate of tumor cells comprising administering to a patient in need thereof, a composition of claim 1.

47. A method of inhibiting growth and/or replication of leukemia cells comprising administering to a patient in need thereof, a composition of claim 1.

48. A method of increasing the mortality rate of leukemia cells comprising administering to a patient in need thereof, a pharmaceutical composition of claim 1.

49. A method of increasing the susceptibility of diseased cells to damage by anti-disease agents comprising administering to a patient in need thereof, a composition of claim 1.

50. A method of increasing the susceptibility of tumor cells to damage by anti-cancer agents comprising administering to a patient in need thereof, a composition of claim 1.

51. A method of increasing the susceptibility of leukemia cells to damage by anti-cancer agents comprising administering to a patient in need thereof, a composition of claim 1.

52. A method of inhibiting increase in number of tumor cells in a patient having a tumor comprising administering to a patient in need thereof, a composition of claim 1.

53. A method of decreasing number of tumor cells in a patient having a tumor comprising administering to a patient in need thereof, a composition of claim 1.

54. A method of inhibiting increase in number of leukemia cells in a patient having leukemia comprising administering to a patient in need thereof, a composition of claim 1.

55. A method of decreasing number of leukemia cells in a patient having leukemia comprising administering to a patient in need thereof, a composition of claim 1.

56. A method of inhibiting cell-cell, cell-matrix, platelet-matrix, platelet-platelet, and/or cell-platelet complex formation comprising administering to a patient in need thereof a composition of claim 1.

57. A method of inhibiting cell-cell, cell-matrix, platelet-matrix, platelet-platelet, and/or cell-platelet aggregation comprising administering to a patient in need thereof a composition of claim 1.

58. A method of inhibiting cell-cell, cell-matrix, platelet-matrix, platelet-platelet, and/or cell-platelet adhesion comprising administering to a patient in need thereof a composition of claim 1.

59. A method of ameliorating the effects of a disease, preventing a disease, treating a disease, or inhibiting the progress of a disease comprising administering to a patient in need thereof a composition of claim 1.

60. A method of therapeutic treatment comprising administering to a patient in need thereof
(i) an antibody, or fragment thereof, and
(ii) an agent,
wherein one or both of the antibody, or fragment thereof, and/or the agent is administered in a sub-clinical amount.

61. The method of claim 60, wherein the antibody, or fragment thereof, and the agent are administered separately.

62. The method of claim 61, wherein the antibody, or fragment thereof, is administered prior to the agent.

63. The method of claim 61, wherein the antibody, or fragment thereof, is administered subsequent to the agent.
